(19) United States
(12) Patent Application Publication
(10) Pub. No.: US 2007/0212341 A1
(43) Pub. Date: Sep. 13, 2007

(54) Method for Preparing Active Kinases Containing Ph Domains

(76) Inventors: Mathias Schmidt, Konstanz (DE); Thomas Beckers, Konstanz (DE)

Correspondence Address: NATH & ASSOCIATES PLLC, 112 South West Street, Alexandria, VA, 22314, US

(21) Appl. No.: 11/658,152

(22) PCT Filed: Jul. 28, 2005
(86) PCT No.: PCT/EP05/53687

§ 371 Date: Jan. 23, 2007

(30) Foreign Application Priority Data
Jul. 30, 2004 (EP) 04103693.0

(51) Int. Cl. A61K 38/48; A61K 48/00; C07H 21/04; C12P 21/06; C12N 9/12

(52) U.S. Cl. 424/94.5; 514/44; 435/69.1; 435/194; 435/320.1; 435/348; 536/23.2

(57) ABSTRACT

The invention relates to a method for preparing active kinases which, in native form, contain a pH domain.

### TECHNICAL FIELD

The present invention relates to a method for preparing active kinases which, in native form, contain a PH domain.

### PRIOR ART

A deregulation of kinases can play a causal role in the origin, progression or pathophysiology of a disease. Kinases are consequently an established enzyme class for finding specific inhibitors which can be used against these diseases. A basis for finding specific inhibitors is that of establishing a biochemical method which can be used to measure the activity of a kinase against a defined substrate. Because kinases are regulated in a complex manner within a cell, expressing a kinase as a recombinant enzyme in heterologous systems, and obtaining it in adequate activity, is frequently very problematical.

An example which has been thoroughly investigated is that of kinases which possess what are termed pleckstrin homology (PH) domains [DiNitto, J. P. Cronin, T. C., Lambright, D. C.: Membrane recognition and targeting by lipid-binding domains. Sci STKE. (213), 1-15 (2003)]. These domains are responsible for locating kinases at the cell membrane, where they can be activated by other enzymes, i.e. what are termed activating kinases.

The kinase B/Akt, which plays a role in oncological diseases, in particular, by mediating signals which are important for the survival of the tumour cell, is a prominent example of a PH domain-regulated kinase. Following activation by growth factors, phosphatidylinositol 3,4,5 trisphosphate (PIP3) is formed proceeding from receptor tyrosine kinases by way of what is termed the PI3 kinase (=phosphatidylinositol-3-kinase). PIP3 is an anchor for the translocation of PH domain-containing proteins such as Akt. Akt is consequently anchored to the cell membrane by way of its PH domain and, in that location, phosphorylated and activated by an activating kinase (in this specific case by the kinase PDK1). After having been activated, Akt translocates from the membrane into the cytoplasm, where it recognizes its substrates and phosphorylates them.

Two functions have consequently been reported to be possessed by the Akt PH domain: (1) anchoring of Akt to the membrane as a prerequisite for activation, and (2) maintaining Akt in an inactive state prior to activation, with the PH domain “covering” the enzymatically active kinase domain (Scheid and Woodgeft (2003) FEBS Letters 27349: 1-5).

As a consequence of the regulation of the activation of Akt by way of the PH domain being complex, the expression of the recombinant enzyme, and its activation, constitute a molecular biological and biochemical problem.

Three methods for generating recombinant, active Akt enzyme have thus far been disclosed:

1. Using recombinant baculoviruses to express an Akt derivative (=truncated Akt protein) which does not possess any PH domain in SF9 (SF=Sfodoptera frugiperda) cells.

2. Expressing recombinant Akt having a H is or GST tag in SF9 cells and purifying the recombinant protein. The protein is subsequently incubated together with PIP3-containing lipid vesicles and PDK1 and the activated protein is purified by means of nickel affinity chromatography [Cohen et al. (1997) FEBS Letters 410: 3; Alessi et al. (1997) Current Biology 7: 777; Alessi et al. (1998) Current Biology 8: 69].

3. Expressing a fusion protein composed of Akt (Akt which contains the PH domain) and an N-terminal glutathione-S-transferase.

A disadvantage in the case of method 1 is the relatively low activity of the Akt enzyme which is generated. A disadvantage in the case of method 2 is the extremely elaborate methodology involving downstream purification procedures. A disadvantage in the case of method 3 is the formation of Akt only some of which is active and which exhibits inhomogeneous phosphorylation patterns [Fabbro et al. (1999) Prot. Expr. Purif. 17: 83-88].

### DESCRIPTION OF THE INVENTION

The object underlying the invention is to provide a simplified method for preparing recombinant Akt enzyme.

The object is achieved by means of the method according to claim 1 and claims 12-14. Preferred embodiments are specified in the subclaims.

It has been found, surprisingly, that recombinant, active Akt enzyme can be prepared, in quantities which are sufficient for biochemical investigation methods and without any elaborate purification procedures, if a truncated PDK1 kinase (=activating kinase) which does not possess any PH domain is coexpressed in an organism or expression system alongside an Akt derivative which likewise does not contain any functional PH domain.

This is particularly surprising to the skilled person since he knew from the prior art that truncated Akt protein, which does not possess any PH domain, can only be expressed with loss of activity in SF9 cells and he would also expect that the quantitative yield of recombinant Akt would decrease when Akt and PDK1 were recombinantly expressed in one organism/expression system.

The present method makes it possible to prepare
Akt kinase in active form, i.e. phosphorylated at threonine 308 and serine 473,
without any elaborate purification procedures, and
in quantities which are sufficient for biochemical investigation methods.

The phosphorylation pattern of active Akt can be established by means of immunoblotting methods using phosphospecific antibodies which are directed against phosphothreonine 308 and phosphoserine 473 (obtainable from Cell Signaling Technology Beverly, Mass.; # 9275 and # 9271). The activity can be measured, for example, by means of a kit from Cell Signaling Technology Beverly, Mass. (# 9840), which can be used to detect the phosphorylation of a GSK3 fusion protein by Akt in an immunoblotting method.

The invention consequently relates to a method for preparing an active kinase derivative which, in native form, contains a PH domain, which method comprises the steps of (a) expressing the kinase derivative which, in native form, contains a PH domain, and (b) coexpressing a derivative of an activating kinase, which derivative, in native form, contains a PH domain, characterized in that the kinase derivative and the derivative of the activating kinase do not contain any functional PH domain.

The active kinase derivative which is generated by means of the present method does not comprise any functional PH domain. No disadvantages ensue from this as far as biochemical investigation methods are concerned since, in a biochemical investigation method, the active enzyme does not need any functional PH domain in order to bring about the phosphorylation of a substrate.

Table 1 specifies, by way of example, some kinases which comprise a PH domain and whose derivatives can be prepared in accordance with the present method. The table also lists the corresponding activating kinases.

According to the present invention, the term “derivative of a kinase” refers to a kinase which is derived structurally from the native form, which possesses an amino acid sequence which is similar to that of the native kinase and which has the enzyme function of the native kinase. Preference is given to derivatives of kinases which, in native form, comprise a PH domain, with the PH domain being altered by point mutation or deletion. Particular preference is given to derivatives of the kinase Akt which do not exhibit any functional PH domain. Very particular preference is given to those Akt kinase derivatives in which the PH domain is deleted.

According to the present invention, the term “PH domain” refers to a pleckstrin homology domain as described in DiNitto, J. P. et al. [DiNitto, J. P. Cronin, T. C., Lambright, D. C.: Membrane recognition and targeting by lipid-binding domains. Sci STKE. (213), 1-15 (2003)].

According to the present invention, the term “active derivative of a kinase” refers to a derivative of a kinase, which derivative exercises the enzyme function of the native kinase. Mention may be made, by way of example, of the function of phosphorylating known natural substrates such as GSK3β, Bad, caspase-9, p27Kip1, NOS or FKHR.

The kinase activity of an active Akt kinase derivative can be detected using a kit from Cell Signaling Technology Beverly, Mass. (#9840). In this assay, the phosphorylation of GSK3 fusion protein by Akt is detected using a phosphospecific antibody.

Another possibility of detecting Akt activity is that of measuring, in a reaction mixture consisting of 100 ng of Akt, 50 mM Hepes (pH 7.5), 3 mM MgCl₂ and 3 mM MnCl₂, 3 μM sodium vanadate, 1 mM DTT, 3 μM ATP and 10 μCi [³³P]-ATP, the phosphorylation of the substrate (histone 2B or GST-GSK3β), by means of a phosphoimager or autoradiography, after carrying out a fractionation by means of polyacrylamide gel electrophoresis and then fixing the gel in 10% acetic acid and drying it.

Another possibility of detecting the enzymic activity of Akt is that of incubating 100 ng of Akt, at 30° C. for 80 min, with 100 ng of histone 2B in a buffer composed of 50 mM Hepes (pH 7.5), 3 mM MgCl₂ and 3 mM MnCl₂, 3 μM sodium vanadate, 1 mM DTT, 1% DMSO, 100 nM ATP and 0.16 nM [³³P]-ATP. After stopping the reaction with phosphoric acid and washing with water, the incorporated cpm [counts per minute] are then determined, in a flashplate (NEN) in a Wallac Trilux measuring instrument, as a measure of the phosphorylation of the histone 2B. In this measuring instrument, the quantity of light which was generated from the radioactive decay by the scintillation gel present in the wells is measured quantitatively. It is a direct measure of the degree of phosphorylation of the Akt substrate employed.

According to the present invention, the term “native form” refers to the naturally occurring form of the kinase. In the case of Akt1, this is the form described under NM.005163 in the NCBI Genbank. In the case of Akt2, this is the form which is described under NM.001626 in the NCBI Genbank. In the case of Akt3, this is the form described under NM.005465 in the NCBI Genbank.

The present method is suitable for recombinantly preparing active kinase derivatives which contain, in their native form, a PH domain and which therefore are to be prepared in an elaborate manner in accordance with the methods described in the prior art. The method is suitable, in particular for preparing active derivatives of the kinases selected from the group comprising Akt1 (NCBI Genbank, NM.005163), Akt2 (NCBI Genbank, NM.001626), Akt3 (NCBI Genbank, NM.005465, NT.004836), β-adrenergic receptor kinases, BtK (NCBI Genbank, NM 000061), LtK (NCBI Genbank, NM.002344), and Tec kinases (NCBI Genbank, NM.003215). The method according to the invention is very particularly suitable for preparing active derivatives of the kinase Akt1, Akt2 or Akt3. In a preferred manner, the present method is suitable for preparing an active Akt1 derivative which does not comprise any functional PH domain.

According to the present invention, the term “no functional PH domain” refers to a functionless PH domain. Using molecular biology, a functionless PH domain can be prepared by carrying out a point mutation in the nucleic acid chain of the PH domain, by means of replacing or deleting more than one nucleotide in the nucleic acid chain of the PH domain or by means of deleting the entire PH domain.

Kinase derivatives which are particularly preferred and which can be prepared using the present method are Akt1ΔPH (=truncated Akt1 protein which does not comprise any PH domain), Akt2ΔPH (=truncated Akt2 protein which does not comprise any PH domain) and Akt3ΔPH (=truncated Akt3 protein which does not comprise any PH domain).

According to the present invention, the term “activating kinase” refers to a kinase which is able to activate another protein by means of phosphorylation. Examples of activating kinases are PDK1 (NCBI Genbank, NM.002613, NM.031268), PKCs (protein kinase C) and src (NCBI Genbank, NM.004383).

TABLE 1
Activating kinases and corresponding activated kinases

| ACTIVATING KINASE | ACTIVATED KINASES |
|---|---|
| PDK1 | Akt1 |
| PDK1 | Akt2 |
| PDK1 | Akt3 |
| src | β-adrenergic receptor kinases |
| PKCs | BtK, ItK, Tec kinases |

According to the present invention, the term “derivative of an activating kinase” refers to a derivative which is derived structurally from the native form of an activating kinase, which has an amino acid sequence which is similar to that of the native activating kinase and which has the enzyme function of the native activating kinase. An example of a derivative of an activating kinase is a derivative of the kinase PDK1 which does not possess any functional PH domain. Another example is a PDK1 derivative in which the PH domain is deleted.

According to the present invention, the term “expression” refers to the transcription of a protein-encoding deoxynucleic acid strand into a protein-encoding ribonucleic acid strand with subsequent translation of this strand into a polypeptide or protein having an amino acid sequence whose coding is predetermined in the deoxyribonucleic acid strand.

According to the present invention, the term “coexpression” refers to the joint expression of two or more proteins in one compartment, for example in an organism or in a noncellular expression system.

According to the present invention, the term “organism” refers to a prokaryotic or eukaryotic organism which is suitable for expressing homologous or heterologous proteins such as kinases and their derivatives. Prokaryotic cells of Escherichia coli, listerias or Bacillus subtilis may be mentioned in this connection by way of example. Eukaryotic cells of Pichia pastoris or P. methanolica, as well as Saccharomyces cerevisiae or S. pombe, or Sfodoptera frugiperda cells, and also mammalian cell systems, such as CHO or HeLa, are likewise suitable. The baculovirus expression system [Baculovirus Expression System Manual, 6th Edition, May 1999, Pharmingen, San Diego, Calif., USA; Baculovirus Expression Vectors, Laboratory Manual, David R. O'Reilly, Lois K. Miller, Verne A. Luckow, WH Freeman & Company, New York, 1992] may be mentioned as being particularly preferred.

According to the present invention, the term “cell-free expression system” refers to a reaction system to which all the components which are required for expressing a protein have been added. In contrast to other expression systems, cell-free expression systems are free from organisms whose endogenous enzymes and cofactors are responsible for expressing heterologous proteins. Examples of cell-free expression systems are the Expressway™ Plus expression system supplied by Invitrogen (Carlsbad, Calif., USA) or the reticulocyte lysate system supplied by Roche Diagnostics (Mannheim, Germany).

Particularly advantageous results are achieved when the method is carried out using a baculovirus expression system. In this connection, a cDNA which [lacuna] a derivative of a kinase which, in native form, contains a PH domain and a cDNA which [lacuna] a derivative of an activating kinase which, in native form, contains a PH domain are prepared using molecular biological methods and cloned into a vector plasmid. The vector plasmids are cotransfected with baculovirus DNA into insect cells. Viruses which encode the derivative of the kinase and the derivative of the activating kinase can be isolated from the supernatant from the transfected insect cells. The isolated viruses can be used to infect insect cells; an active derivative of a kinase which, in native form, contains a PH domain can then be isolated from the supernatant when the insect cells are incubated. The advantageous method is particularly well suited for preparing an active derivative of the kinase Akt1 which does not possess any functional PH domain.

According to the present invention, the term “cloning” refers to the insertion of nucleic acid sequences into a vector or a plasmid and the replication of the latter in a suitable organism.

According to the present invention, the term “cotransfection” refers to the insertion of several nucleic acids into a cell of an expression system. Particular preference is given to cotransfecting BaculoGold baculovirus DNA (Pharmingen, San Diego, Calif., USA) with vectors which are compatible with this system and contain the nucleic acid sequence which encodes an activating kinase derivative or a kinase derivative which is to be activated.

According to the present invention, the term “insect cells” refers to cells which are derived from some developmental form (egg, larva, pupa or imago) of the order Hexapoda. Immortal cells derived from Sfodoptera frugiperda, such as, for example, SF9, SF21 or SF158, are particularly suitable.

According to the present invention, the term “infection” refers to the incubation of cells with viruses or phages which possess the ability to be taken up into the cell, to replicate in the cell and to induce, in the cell, the expression of a heterologous, virus-encoded protein.

According to the present invention, the term “incubation” refers to maintaining a cell culture, sample or reaction mixture at a given temperature for a given time.

According to the present invention, the term “isolating viruses from the culture supernatants of the insect cells” refers to the removal of culture supernatants from insect cells which are infected with viruses and secrete viruses or which are cotransfected with virus DNA and vector DNA for the purpose of generating recombinant viruses. The isolated viruses are suitable for being stored or for further infections.

According to the present invention, the term “obtaining the active kinase derivative” refers to the isolation of the activated kinase derivative and its preparation in enriched form by separating off the activating kinase and other constituents of the expression system. This can be achieved by a variety of methods, for example by means of affinity chromatography using glutathione-coated or nickel-coated matrices, by means of ion exchange chromatography, by means of molecular sieve chromatography, by means of hydrophobic interaction chromatography, by means of affinity chromatographies using ligands, chromophores, lipids or antibodies, by means of protein precipitation or by means of different centrifugation methods. The active kinase derivative which is obtained can then be made storable at −80° C. by adding additives such as 20% glycerol.

The present invention furthermore relates to the use of an active Akt derivative, which can be obtained by a method according to the invention, in a biochemical investigation method for identifying Akt1 inhibitors. In particular, an Akt1 derivative having a deleted PH domain is particularly suitable for being used in such an assay. The method described in Example 6 for identifying Akt1 inhibitors is mentioned here by way of example.

### INDUSTRIAL APPLICABILITY

The method according to the invention makes it possible to prepare active Akt1 in quantities which are sufficient for biochemical methods. As compared with the methods described in the prior art, the method according to the invention enjoys the following advantages:
markedly higher activity of Akt1 which is prepared using the method
lower complexity of the individual implementation steps
higher yields, since only one purification step is required
more homogeneous preparation of the enzyme fraction
lower batch variability as a consequence of a more marked reduction in the complexity of the enzymic activation steps.

The active kinases, such as active Akt1ΔPH, which can be obtained using the method according to the invention are suitable for being used in biochemical test methods and consequently for finding specific inhibitors [lacuna] in which these kinases play a pathophysiological role.

### DESCRIPTION OF THE FIGURES

FIG. 1a: Determining the Enzymic Activity of Different Akt Preparations in Regard to Histone 2B as Substrate

The figure depicts the result of a kinase activity measurement performed as described in Ex. 4a. In this example, truncated Akt1 protein which does not comprise any PH domain was expressed in SF9 cells, using the baculovirus system, and then purified (results of the activity measurement in right-hand columns). Coinfection was also used to express truncated Akt1 protein, which does not comprise any PH domain, and truncated PDK1 protein, which does not comprise any PH domain, in SF9 cells using the baculovirus system, after which these proteins were purified (results of the activity measurement in left-hand columns). For comparison, Akt1 (possessing a PH domain and possessing two activating mutations at positions 308 and 473) was expressed in SF9 cells and then purified (results of the activity measurement in central columns). It is to be noted that the Akt derivative which does not possess a PH domain and which is activated by coinfection with PDK1 (likewise without a PH domain) (left-hand columns) displays a markedly higher activity than does Akt1ΔPH (=truncated Akt1 protein which does not comprise any PH domain) which is not activated by coinfection with PDK1-encoding baculoviruses (right-hand columns). PDK1-activated Akt1ΔPH (left-hand columns) also possesses a markedly higher activity than the Akt kinase derivative which contains the PH domain and in which the amino acids threonine 308 and serine 473 are replaced with aspartic acid (central columns).

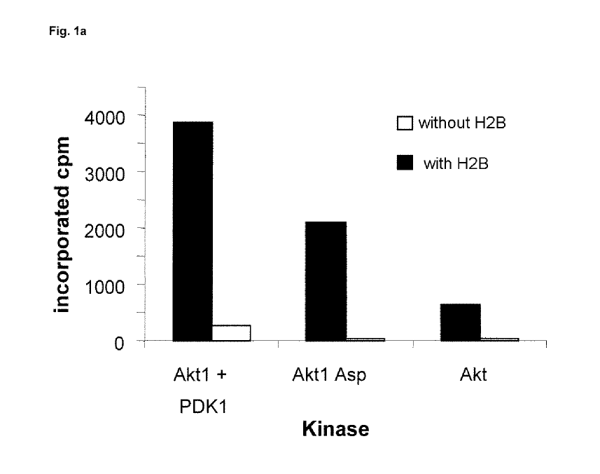

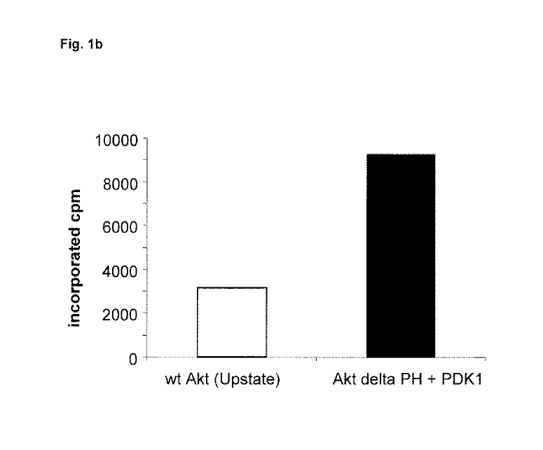

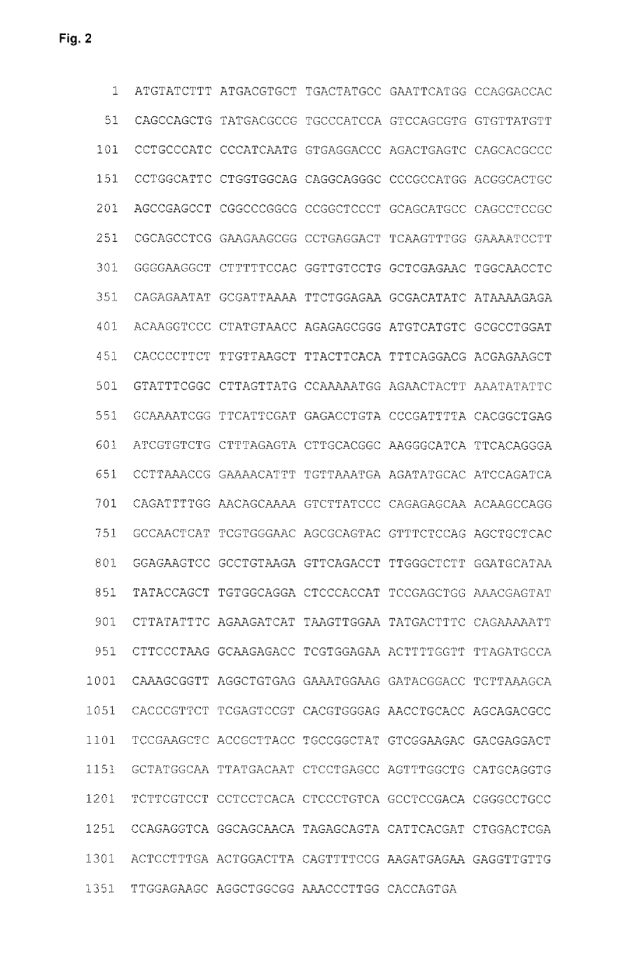

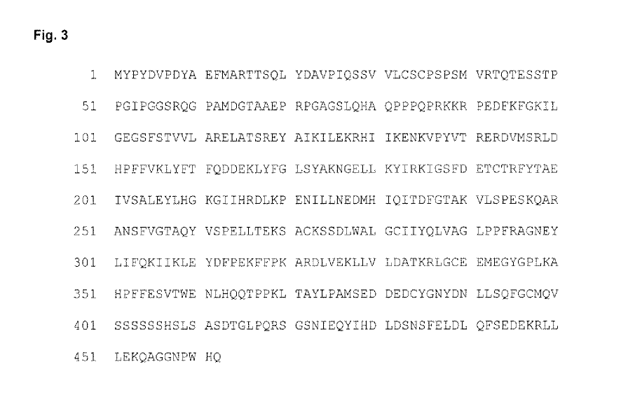

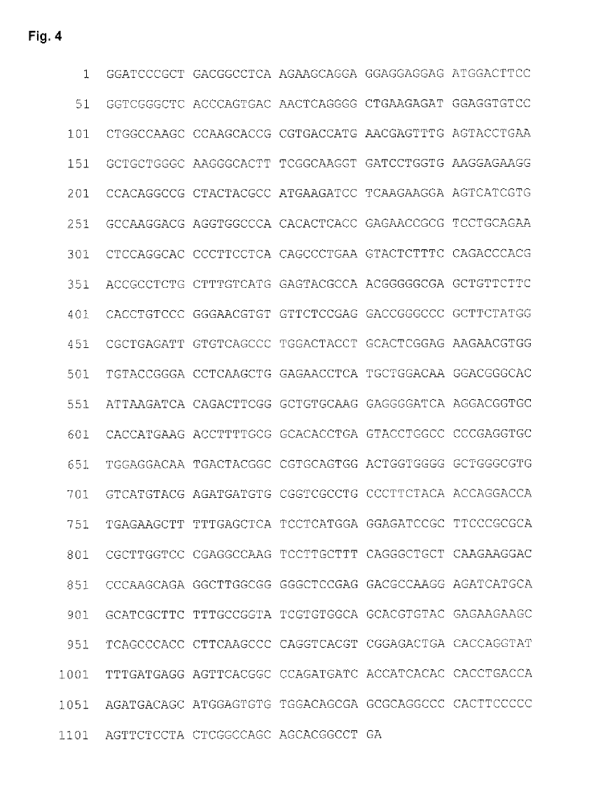

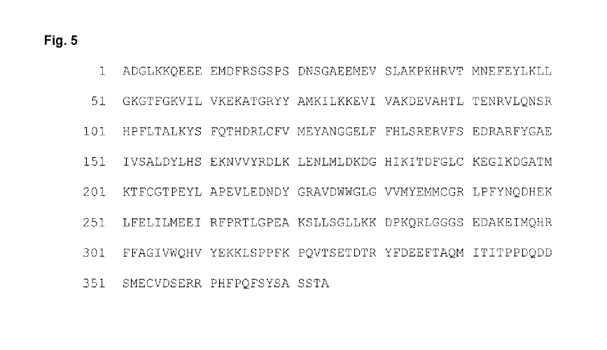

FIG. 1b: Comparing the Enzymic Activities of Different Akt Variants in Regard to Histone 2B as the Substrate

The figure depicts the result of a kinase activity measurement which was performed as described in Ex. 4b. In this example, truncated Akt1 protein, which does not comprise any PH domain, is expressed in SF9 cells, using the baculovirus system, and then purified (results of the activity measurement in the right-hand column). Commercially obtainable wild-type Akt1 protein (Upstate Biochemicals, Charlottsville, Va., USA) which was likewise expressed in SF9 cells using the baculovirus system and then purified, is also used (results of the activity measurement in the left-hand column). It is to be noted that the Akt derivative without a PH domain, which is activated by coinfection with PDK1 (likewise without a PH domain) (right-hand columns) displays a markedly higher activity than wild-type Akt1, which is not activated by coinfection with PDK1-encoding baculoviruses (left-hand column).

FIG. 2: Truncated PDK1 Sequence

The figure depicts the DNA which encodes PDK1 amino acids 1 (ATG) to 450 (CAG). The DNA encoding amino acid 451 through to the natural stop codon was deleted and replaced with a TGA stop codon after the DNA sequence for amino acid 450.

FIG. 3: Amino Acid Sequence Deduced from the Nucleic Acid Sequence Shown in FIG. 2

The figure depicts the encoded amino acids 1 (ATG) to 450 (CAG) for PDK1. The DNA encoding amino acid 451 through to the natural stop codon was deleted and replaced with a TGA stop codon after the [lacuna] for amino acid 450.

FIG. 4: Truncated Akt1 Sequence

The figure depicts the DNA which encodes Akt1 amino acids 107 (gct) to 481 (gcc). The first 7 nucleotides are sequences from the vector pAcG1 (Pharmingen, San Diego, Calif., USA) containing the BamHI restriction cleavage site by way of which the DNA sequence encoding the Akt1 delta PH was inserted into the vector. The concluding TGA sequence is the stop codon for terminating the protein translation.

FIG. 5: Amino Acid Sequence Deduced from the Nucleic Acid Sequence Shown in FIG. 4

The figure depicts the amino acid sequence for Akt1 amino acids 107 (A) to 481 (A) of the native Akt1 kinase.

### EXAMPLES

### Ex. 1

### Preparing the PDK1 Construct which Encodes a Truncated PDK1

In order to express PDK1 having a deleted PH domain (=PDK1ΔPH; truncated PDK1 protein which does not comprise any PH domain), RNA was isolated from RKO human colon adenocarcinoma cells using the Qiagen RNeasy kit (Qiagen, Hilden, Germany). The RNA was reverse-transcribed into cDNA using the Roche cDNA amplification kit (Roche Diagnostics, Mannheim, Germany). Parts of this cDNA which encode amino acids 1-450 from native PDK1 were amplified by means of PCR and using specific primers. In the PCR, a sequence tag [V5 tag], for detecting the recombinant protein, and a TGA stop codon were also inserted at the 5′ end of the PCR amplificate. The amplificate was cloned into the vector pVI1392 (Pharmingen, San Diego, Calif.) using the restriction cleavage sites NotI and BamHI which were inserted during the PCR. The cloning result is depicted in FIG. 2. The corresponding amino acid sequence is depicted in FIG. 3.

### Ex. 2

### Preparing the Akt1 Construct which Encodes a Truncated Akt1

In order to express Akt1 possessing a deleted PH domain (=Akt1ΔPH, truncated Akt1 protein which does not comprise any PH domain), RNA was isolated from RKO human colon adenocarcinoma cells using the Qiagen RNeasy kit (Qiagen, Hilden, Germany). The RNA was reverse-transcribed into cDNA using the Roche cDNA amplification kit (Roche Diagnostics, Mannheim, Germany). Parts of this cDNA which encode amino acids 107-481 from native Akt1 were amplified by means of PCR using specific primers. The primers employed were used to insert a BamHI restriction cleavage site and an EcoRI restriction cleavage site. The cDNA was cloned into the vector pAcG1 (Pharmingen, San Diego, Calif.) using these restriction cleavage sites such that the reading frame containing the N-terminal, vector-encoded GST domain was preserved. The sequence used for the cloning (beginning at the BamHI cleavage site) is depicted in FIG. 4. The corresponding amino acid sequence is depicted in FIG. 5.

### Ex. 3

### Preparing Active, Truncated Akt1

In order to generate recombinant baculoviruses, the cDNAs encoding GST-Akt1ΔPH (=cDNA encompassing truncated akt1 which has a GST tag and whose PH domain is deleted; nucleic acid sequence, see FIG. 4; amino acid sequence, see FIG. 5) and PDK1ΔPH (=cDNA encompasses truncated pdk1 whose PH domain is deleted; nucleic acid sequence, see FIG. 2; amino acid sequence, see FIG. 3) were cotransfected with Baculo Gold DNA (Pharmingen, San Diego, Calif.) into SF9 cells. 6 days after transfection, the culture supernatants containing recombinant baculoviruses were harvested, sterilized by filtration and in each case amplified twice for 5 days in SF9 cells by adding 300 μl of virus supernatant to a T162 cell culture flask which contained 6×10⁷ SF9 cells. In order to express active recombinant Akt1 kinase, 100 μl of virus supernatant of the PDK1-encoding baculoviruses and 300 μl of the Akt1-encoding baculoviruses were added to 6×10⁷ SF9 cells and the culture was incubated at 27° C. for three days. The cells were then harvested and lysed in lysis buffer (50 mM Tris, pH 7.5, 1% NP40, 1 mM PMSF, 1 mM EDTA, 100 μM NaVO₄, 20 mM NaF, 5 mM DTT) and homogenized and insoluble fragments were centrifuged off. The lysate was loaded onto a chromatography column containing glutathione-Sepharose 4B (Amersham Biosciences). After the column had been washed thoroughly with lysis buffer with and without detergent, specifically bound protein was eluted with elution buffer (50 mM Tris, pH 8, 10 mM glutathione (reduced); 50 mM NaCl, 2 mM DTT, 10 μM NaVO₄). The phosphorylation of Akt1 at threonine 308 and Ser 473 was determined in a Western blot by means of phosphospecific antibodies (Cell Signaling Technology, Beverly, Mass.) using 50 ng of purified recombinant protein.

### Ex. 4a

### Measuring the Kinase Activity

In order to measure the kinase activity of truncated Akt1 (Akt1ΔPH) as well as of native Akt1, in each case 100 ng of Akt were incubated, at 30° C. for 80 min, with 100 ng of histone 2B in a buffer comprising 50 mM Hepes, pH 7.5, 3 mM MgCl₂ and 3 mM MnCl₂, 3 μM sodium vanadate, 1 mM DTT, 1% DMSO, 100 nM ATP and 0.16 nM [³³P]ATP. After the reaction had been stopped with phosphoric acid and the wells had been washed with water, the incorporated flashplate cpm [counts per minute] were then determined in a (N EN), in a Wallac Trilux measuring instrument, as a measure of the histone 2B phosphorylation. In this measuring instrument, the quantity of light which was generated from the radioactive decay by the scintillation gel which was present in the wells was measured quantitatively. It is a direct measure of the degree of the phosphorylation of the Akt substrate employed.

### Ex. 4b

### Comparing the Enzymic Activity of Different Akt Variants in Regard to Histone 2B as Substrate

Truncated Akt1 protein, which does not comprise any PH domain, was expressed in SF9 cells using the baculovirus system and then purified and a kinase activity measurement as described in Ex. 4a was carried out (results of the activity measurement in FIG. 1a, right-hand column). Commercially obtainable wild-type Akt1 protein (Upstate Biochemicals, Charlottsville, Va., USA), which was likewise expressed in SF9 cells using the baculovirus system and then purified, was also used (results of the activity measurement in FIG. 1a, left-hand column).

It is to be noted that the Akt derivative without PH domain, which derivative is activated by coinfection with PDK1 (likewise without PH domain) (right-hand columns) displays a markedly higher activity than wild-type Akt1 which is not activated by coinfection with PDK1-encoding baculoviruses (left-hand column).

### Ex. 5

### Activity Test Performed on an Activated Akt Derivative

The activity of an Akt derivative, which was expressed using the present method and obtained as an active kinase derivative, was compared with the activity of a commercially obtainable Akt1 enzyme (Upstate, Charlottsville, Va., USA). The activity of the commercially obtainable enzyme was obtained by expressing the enzyme, which contained the PH domain, in the baculovirus system and then purifying it. The enzyme was subsequently incubated with PDK1 in the presence of phosphatidylinositol 3,4,5-phosphate-containing lipid vesicles in order to phosphorylate Akt1 at threonine 308. After that, the activated Akt enzyme was separated chromatographically in order to separate it off from the activating PDK1 kinase. The enzyme activities of the two kinases were measured as described in Ex. 4. It was found that Akt1ΔPH, which was obtained by coinfecting with PDK1ΔPH-encoding baculoviruses, possessed at least the same activity as native Akt1 which was activated by subsequent phosphorylation by PDK1 in the presence of lipid vesicles.

### Ex. 6

### Identifying an Akt1 Inhibitor

The ability of an Akt1 inhibitor to inhibit Akt1 activity was determined as a measure of the activity of the inhibitor. For this, the kinase activity of truncated Akt1 (Akt1ΔPH) was measured with and without inhibitor. In this connection, in each case 100 ng of Akt1ΔPH were incubated, at 30° C. for 80 min, with 100 ng of histone 2B in a buffer composed of 50 mM Hepes, pH 7.5, 3 mM MgCl₂ and 3 mM MnCl₂, 3 μM sodium vanadate, 1 mM DTT, 1% DMSO, 100 nM ATP and 0.16 nM [³³P]ATP. The inhibitor to be investigated was added to the mixture at different concentrations in 1% DMSO. After stopping the reaction with phosphoric acid and washing the wells with water, the incorporated cpm [counts per minute] were then determined in a flashplate (NEN), in a Wallac Trilux measuring instrument, as a measure of the histone 2B phosphorylation. In this measuring instrument, the quantity of light which was generated from the radioactive decay by the scintillation gel which was present in the wells was measured quantitatively. It is a direct measure of the degree of phosphorylation of the Akt substrate employed. Substances which inhibited the ability of Akt1 to phosphorylate its substrate were identified as being Akt1 inhibitors.

1. A method for preparing an active derivative of a kinase which, in native form, contains a PH domain, comprising the steps of
expressing a derivative of a kinase which, in native form, contains a PH domain, and
coexpressing a derivative of an activating kinase which, in native form, contains a PH domain,
characterized in that the derivative of the kinase and the derivative of the activating kinase do not contain any functional PH domain.

2. The method according to claim 1, characterized in that the expression of the derivative of the kinase and the coexpression of the derivative of the activating kinase take place in the same organism.

3. The method according to claim 2, characterized in that the expression takes place in a baculovirus expression system.

4. The method according to claim 1, characterized in that the expression of the derivative of the kinase and the coexpression of the derivative of the activating kinase take place in a cell-free expression system.

5. The method according to claim 1, comprising the steps of
(a) preparing a cDNA which encodes a derivative of a kinase which, in native form, contains a PH domain,
(b) preparing a cDNA which encodes a derivative of an activating kinase which, in native form, contains a PH domain,
(c) cloning the cDNA from steps (a) and (b) into a plasmid,
(d) expressing the kinase derivative and the derivative of the activating kinase, and
(e) isolating the active kinase derivative.

6. The method according to claim 5, comprising the steps of
(a) preparing a cDNA which encodes a derivative of a kinase which, in native form, contains a PH domain,
(b) preparing a cDNA which encodes a derivative of an activating kinase which, in native form, contains a PH domain,
(c) cloning the cDNA from steps (a) and (b) into a plasmid,
(d) cotransfecting cloning products from step (c) and baculovirus DNA into insect cells,
(e) incubating the transfected insect cells,
(f) isolating viruses from the culture supernatants of the insect cells from step (e),
(g) infecting insect cells with the viruses from step (f),
(h) incubating the infected insect cells from step (g), and
(i) isolating the active kinase derivative from the insect cells from step (h).

7. The method according to claim 1 wherein said active derivative of a kinase is selected from the group consisting of one or more of the following:
active Akt1 which does not contain any functional PH domain,
active Akt2 which does not contain any functional PH domain, and
active Akt3 which does not contain any functional PH domain.

8. The method according to claim 1, characterized in that active Akt1 which does not contain any functional PH domain is prepared.

9. The method according to claim 1, characterized in that active Akt1ΔPH, active Akt2ΔPH or active Akt3ΔPH is prepared.

10. The method according to claim 1, characterized in that active Akt1ΔPH is prepared.

11. The method according to claim 1, characterized in that the derivative of an activating kinase which, in native form, contains a PH domain is PDK1ΔPH.

12. An active Akt1 derivative which does not contain any functional PH domain and which is obtained by the method according to claim 1.

13. An active Akt1 derivative which has a deleted PH domain and which is obtained by the method according to claim 1.

14. (canceled)

15. A method for screening for an Akt1 inhibitor, comprising the steps of:
(a) providing an Akt1 derivative produced by the process of claim 1,
wherein, in relation to Akt1 in its native form, said Akt1 derivative either (i) lacks a functional PH domain or (ii) lacks a PH domain;
(b) providing an Akt1 substrate;
(c) mixing said Akt1 derivative, said Akt1 substrate, and a putative Akt1 inhibitor in a reaction mixture suitable for an Akt1 substrate phosphorylation reaction; and
(d) measuring the degree, if any, of Akt1 substrate phosphorylation in said reaction mixture.

16. The method of claim 15, further comprising the step of correlating the degree, if any, of Akt1 substrate phosphorylation with the ability of said putative Akt1 inhibitor to inhibit Akt1 activity.

17. The method of claim 15, further comprising the step of stopping said reaction after step (c) and before step (d).

18. The method of claim 15, further comprising the step of washing said reaction mixture after step (c) and before step (d).


SEQUENCE LISTING (4 sequences)

SEQ ID NO: 1 - DNA, 1389 bp, Homo sapiens
  CDS (1)..(1389): Trunkated PDK1 sequence from Fig. 2
atgtatctttatgacgtgcttgactatgccgaattcatggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagatcgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtga

SEQ ID NO: 2 - protein, 462 aa, Homo sapiens
MYLYDVLDYAEFMARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQ

SEQ ID NO: 3 - DNA, 1132 bp, Homo sapiens
  CDS (8)..(1132): truncated Akt1 sequence from Fig. 4
ggatcccgctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctga

SEQ ID NO: 4 - protein, 374 aa, Homo sapiens
ADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA